(19) United States
(12) Patent Application Publication
(10) Pub. No.: US 2025/0152921 A1
(43) Pub. Date: May 15, 2025

(54) GUIDEWIRE CATHETER DELIVERY DEVICE

(71) Applicant: SHANGHAI OPERATION ROBOT CO., LTD., Shanghai (CN)

(72) Inventors: Wenming LIU, Shanghai (CN); Zhongwei YU, Shanghai (CN); Daozhi LIU, Shanghai (CN); Yikun LIU, Shanghai (CN)

(73) Assignee: SHANGHAI OPERATION ROBOT CO., LTD., Shanghai (CN)

(21) Appl. No.: 18/840,511

(22) PCT Filed: Dec. 21, 2022
(86) PCT No.: PCT/CN2022/140594

§ 371 Date: Aug. 22, 2024

(30) Foreign Application Priority Data
Feb. 23, 2022 (CN) 202210169988.9

(51) Int. Cl. A61M 25/09; A61M 25/01

(52) U.S. Cl. CPC A61M 25/09041; A61M 25/0116; A61M 2205/103; A61M 2205/332

(57) ABSTRACT

A guidewire catheter delivery device includes a guidewire catheter delivery module and a motor drive and force feedback module; the motor drive and force feedback module is drivingly connected to the guidewire catheter delivery module, and the guidewire catheter delivery module is installed with a guidewire catheter and drives the guidewire catheter to move back and forth; a force sensor and a motor are installed in the motor drive and force feedback module; an output end of the motor is connected to the guidewire catheter delivery module, and an end, facing away from the guidewire catheter delivery module, of the motor is connected to the force sensor; a driving wheel and a driven wheel are installed in the guidewire catheter delivery module; and the driving wheel is connected to the output end of the motor, and the guidewire catheter is installed between the driving wheel and the driven wheel.

### CROSS-REFERENCE TO THE RELATED APPLICATIONS

This application is the national phase entry of International Application No. PCT/CN2022/140594, filed on Dec. 21, 2022, which is based upon and claims priority to Chinese Patent Application No. 202210169988.9, filed on Feb. 23, 2022, the entire contents of which are incorporated herein by reference.

### TECHNICAL FIELD

The present invention relates to the field of medical devices and particularly to a guidewire catheter delivery device.

### BACKGROUND

As a very mature endoscopic minimally invasive technique in the treatment of diseases of the biliary pancreatic system, endoscopic retrograde cholangiopancreatography (ERCP) can be used to diagnose and treat gallstones, biliary obstruction, cholangitis, biliary tumors, pancreatic tumors, and the like. During the operation, a duodenoscope is inserted into the descending part of the duodenum of the patient, an angiographic catheter is inserted into the biopsy tube to reach the duodenal papilla opening, and then contrast agent is injected. The specific pancreaticobiliary conditions are observed under the X-ray film to determine whether there were lesions, and then the corresponding operation is carried out. ERCP has the advantages of less trauma, short operation time, few complications, and high safety. ERCP, as minimally invasive surgery, just causes a small surgical trauma, does not bring too much pain to the patient, and has fast postoperative recovery, but it needs to be done with the aid of X-rays, and the doctor for this surgery must be exposed to X-rays for a long time. Therefore, in order to improve the working environment of the doctors for interventional surgery, ERCP surgical robots have been developed in engineering to substitute doctors and complete surgery. As a result, doctors control ERCP surgical robots to complete surgery in an X-ray free environment through remote operation or remote control. At present, there is no effective and similar technical solution on the market.

At present, ERCP operations in China are performed manually by doctors and their teams. During the operation, the operators need to wear heavy anti-radiation suits, but the arm part must be exposed for operation, which cannot be protected against radiation. Long-term surgical radiation can cause serious radiation damage to the operators. In addition, the existing ERCP operation requires a lot of operators and cooperators, so it is crowded in the space-limited operating room. Doctors and operators need to stand all day long for surgery, which causes high-intensity work, and it is easy to fatigue and thus affect the accuracy of surgery and even lead to mistakes. Additionally, during the operation, it is difficult for the doctors and the operators to ensure that the hand does not shake, so the displacement may occur after the inserted human instrument is positioned.

The patent document (CN105664333A) discloses a guidewire catheter delivery device, including a guidewire, a catheter, a guidewire delivery system, and a catheter delivery system. The catheter delivery system includes a catheter delivery guide rail and can move along the axial direction of the catheter delivery guide rail, and the catheter is fixed on the catheter delivery system. The guidewire delivery system includes a guidewire delivery guide rail and can move along the axial direction of the guidewire delivery guide rail, and the guidewire is fixed on the guidewire delivery system. The guidewire delivery guide rail is fixed on the catheter delivery guide rail, and the guidewire is coaxial with the catheter.

The patent document (CN110624171A) discloses a guidewire catheter delivery device, including a guidewire moving clamping mechanism, a guidewire fixing clamping mechanism, a guidewire axial moving mechanism, a catheter delivery clamping mechanism, and a base. The guidewire moving clamping mechanism, the guidewire fixing clamping mechanism, the guidewire axial moving mechanism and the catheter delivery clamping mechanism are respectively provided on the base along the axial feeding direction of the guidewire.

### SUMMARY

Aiming at the shortcomings in the prior art, the object of the present invention is to provide a guidewire catheter delivery device.

According to one or more embodiments of the present invention, a guidewire catheter delivery device includes: a guidewire catheter delivery module and a motor drive and force feedback module;
the motor drive and force feedback module is drivingly connected to the guidewire catheter delivery module, and the guidewire catheter delivery module is installed with a guidewire catheter and drives the guidewire catheter to move back and forth;
a force sensor and a motor are installed in the motor drive and force feedback module;
an output end of the motor is connected to the guidewire catheter delivery module, and an end, facing away from the guidewire catheter delivery module, of the motor is connected to the force sensor;
a driving wheel and a driven wheel are installed in the guidewire catheter delivery module; and
the driving wheel is connected to the output end of the motor, and the guidewire catheter is installed between the driving wheel and the driven wheel.

Further, the motor drive and force feedback module further includes: a motor support frame and a support shaft;
the force sensor and the motor are installed in the motor support frame, the output end of the motor extends out of one end of the motor support frame, and the force sensor is installed at the other end of the motor support frame; and
the support shaft extends outward from a middle of the motor support frame.

Further, the motor support frame is installed in a fixed seat;
the motor support frame is rotatably connected to the fixed seat through the support shaft;
the fixed seat is fixedly connected to the force sensor; and
when the guidewire catheter moves, the motor receives a reaction force from the guidewire catheter, the end, adjacent to the guidewire catheter delivery module, of the motor support frame receives the reaction force, and the end, connected to the force sensor, of the motor support frame receives a force that is opposite to the reaction force through the support shaft.

Further, the guidewire catheter delivery module further includes: a main body, a flip cover, a driven wheel frame, and a bottom cover;
the bottom cover is installed at a bottom of the main body, and the flip cover is rotatably connected to the main body through a shaft pin; the main body, the bottom cover, and the flip cover are surrounded to form a cavity;
the driven wheel frame, the driving wheel, and the driven wheel are installed in the cavity;
the driving wheel is installed on a side of the driven wheel frame, and the driven wheel is installed inside the driven wheel frame;
the driven wheel frame is allowed to drive the driven wheel to move relative to the driving wheel and the main body;
when the flip cover rotates away from the main body through the shaft pin to be opened, the guidewire catheter is allowed to be installed in the main body; and
when the flip cover rotates towards the main body through the shaft pin to be closed, the flip cover limits movement of the guidewire catheter.

Further, the driven wheel is rotatably connected to the driven wheel frame, and the driven wheel is allowed to move towards or away from the driving wheel relative to the driven wheel frame;
a driven wheel pressing ball is installed on a side, facing away from the driving wheel, of the driven wheel frame, and a driven wheel pressing spring is installed between the driven wheel and the driven wheel pressing ball; and
when the flip cover is closed, the driven wheel frame is pressed by the flip cover to move towards the driving wheel, and the driven wheel pressing spring presses the driven wheel towards the driving wheel.

Further, a limit block is installed on a side wall of the driven wheel frame; and
when the driven wheel frame moves away from the driving wheel, the limit block interferes with the main body, and the driven wheel frame limits a distance away from the driving wheel through the limit block.

Further, a boss is provided on a side, adjacent to the driving wheel, of an inner wall of the main body, and a driven wheel release spring is installed between the limit block and the boss; and
when the flip cover is opened, the driven wheel frame moves away from the driving wheel through the driven wheel release spring.

Further, buckles are provided on sides, facing the motor drive and force feedback module, of the flip cover and the bottom cover; and
the buckles are clamped on the motor support frame.

Further, the driven wheel is installed on the driven wheel frame by a screw and a nut; the driven wheel is allowed to rotate around the screw;
the screw and the nut are allowed to move relative to the main body; and
a first bearing and a second bearing are installed between the driving wheel and the main body, and a third bearing is installed between the driven wheel and the screw.

Further, a robot includes the guidewire catheter delivery device.

Preferably and further, the output end of the motor is configured to be a D-shaped shaft;
a side, facing the motor, of the driving wheel is provided with a D-shaped hole; and
the D-shaped shaft is matched with the D-shaped hole.

Further, the buckle is connected to a button and released by pressing the button.

### BRIEF DESCRIPTION OF THE DRAWINGS

Other features, objects, and advantages of the present invention will become more apparent by reading the detailed description of non-limiting embodiments with reference to the following drawings:

FIG. 1 is an exploded view of a guidewire catheter delivery device;

FIG. 2 is a schematic diagram showing the structure of the guidewire catheter delivery device;

FIG. 3 is a front view of the guidewire catheter delivery device;

FIG. 4 is a schematic diagram showing the structure of a guidewire catheter delivery module;

FIG. 5 is a cross-section view of the guidewire catheter delivery module;

FIG. 6 is a cross-section view of the guidewire catheter delivery device;

FIG. 7 is a schematic diagram showing the structure of the guidewire catheter delivery module after the flip cover is opened;

FIG. 8 is a cross-section view of a motor drive and force feedback module;

FIG. 9 is a schematic diagram showing the structure of a screw; and

FIG. 10 is a schematic diagram showing the structure of a nut.

The reference numerals in the drawings are as follows:

| | | | |
|---|---|---|---|
| guidewire catheter delivery module | 1 | bottom cover | 113 |
| main body | 101 | limit block | 114 |
| flip cover | 102 | driven wheel pressing spring | 115 |
| driving wheel | 103 | motor drive and force feedback module | 2 |
| driven wheel | 104 | motor support frame | 201 |
| driven wheel frame | 105 | force sensor | 202 |
| driven wheel release spring | 106 | motor | 203 |
| driven wheel pressing ball | 107 | support shaft | 204 |
| screw | 108 | fixed seat | 205 |
| nut | 109 | guidewire catheter | 3 |
| first bearing | 110 | | |
| second bearing | 111 | | |
| third bearing | 112 | | |

### DETAILED DESCRIPTION OF THE EMBODIMENTS

The present invention will be described in detail below in conjunction with specific embodiments. The following embodiments will help those skilled in the art to further understand the present invention, but do not limit the present invention in any form. It should be pointed out that for those having ordinary skill in the art, several changes and improvements may be made without departing from the ideas of the present invention. These all fall within the protection scope of the present invention.

### Embodiment 1

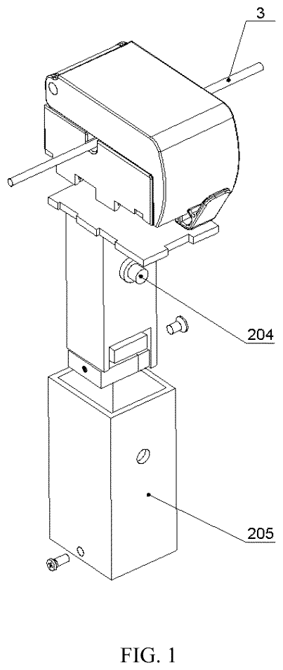

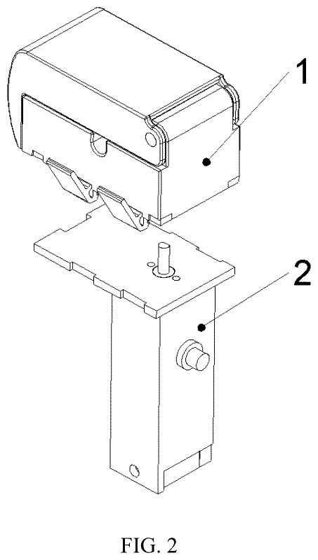

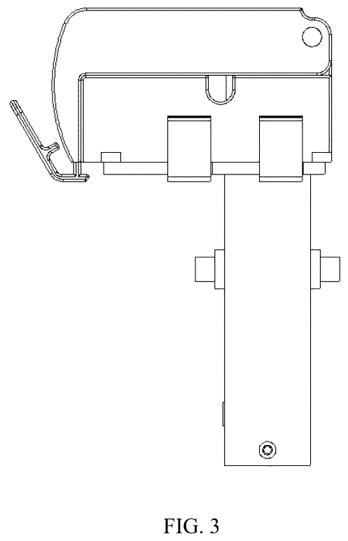

As shown in FIGS. 1 to 3, a guidewire catheter delivery device, which can be used for a robot, includes: the guidewire catheter delivery module 1 and the motor drive and force feedback module 2. The motor drive and force feedback module 2 is drivingly connected to the guidewire catheter delivery module 1, and the guidewire catheter delivery module 1 is installed with the guidewire catheter 3 and drives the guidewire catheter 3 to move back and forth. The force sensor 202 and the motor 203 are installed in the motor drive and force feedback module 2. An output end of the motor 203 is connected to the guidewire catheter delivery module 1, and an end, facing away from the guidewire catheter delivery module 1, of the motor 203 is connected to the force sensor 202. The driving wheel 103 and the driven wheel 104 are installed in the guidewire catheter delivery module 1. The driving wheel 103 is connected to the output end of the motor 203. The guidewire catheter 3 is installed between the driving wheel 103 and the driven wheel 104.

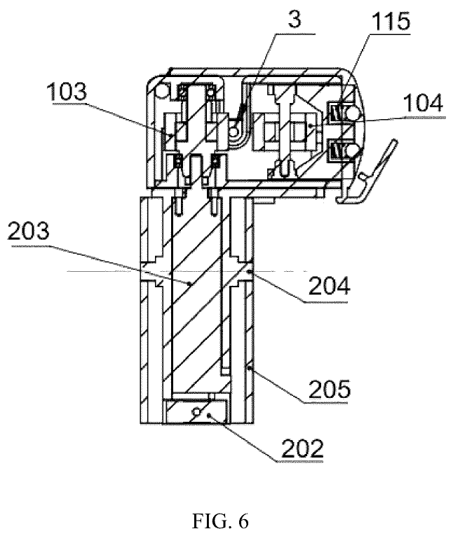

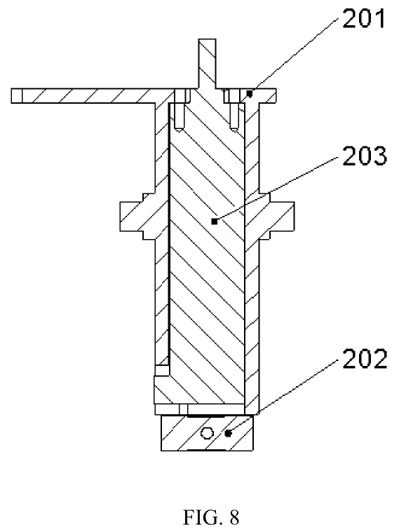

As shown in FIGS. 6 and 8, the motor drive and force feedback module 2 further includes: the motor support frame 201 and the support shaft 204. The force sensor 202 and the motor 203 are installed in the motor support frame 201. The output end of the motor 203 extends out of one end of the motor support frame 201, and the force sensor 202 is installed at the other end of the motor support frame 201. The support shaft 204 extends outward from a middle of the motor support frame 201. The motor support frame 201 is installed in the fixed seat 205, the motor support frame 201 is rotatably connected to the fixed seat 205 through the support shaft 204, and the fixed seat 205 is fixedly connected to the force sensor 202. When the guidewire catheter 3 moves, the motor 203 receives the reaction force from the guidewire catheter 3, the end, adjacent to the guidewire catheter delivery module 1, of the motor support frame 201 receives the reaction force, and the end, connected to the force sensor 202, of the motor support frame 201 receives the force that is opposite to the reaction force through the support shaft 204.

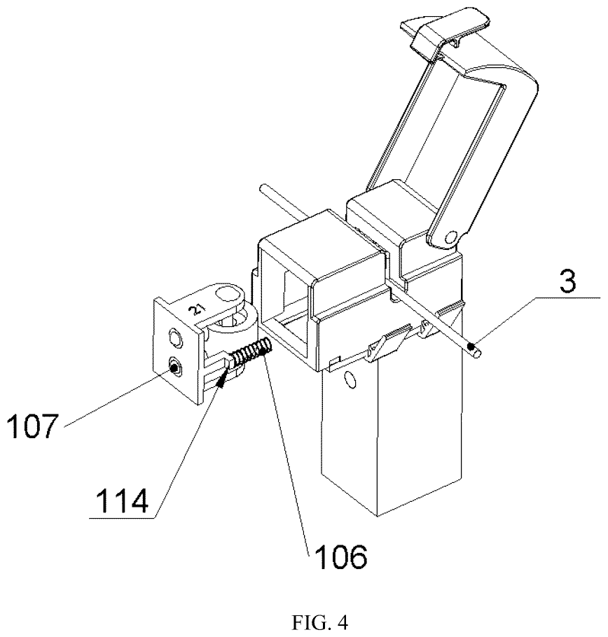

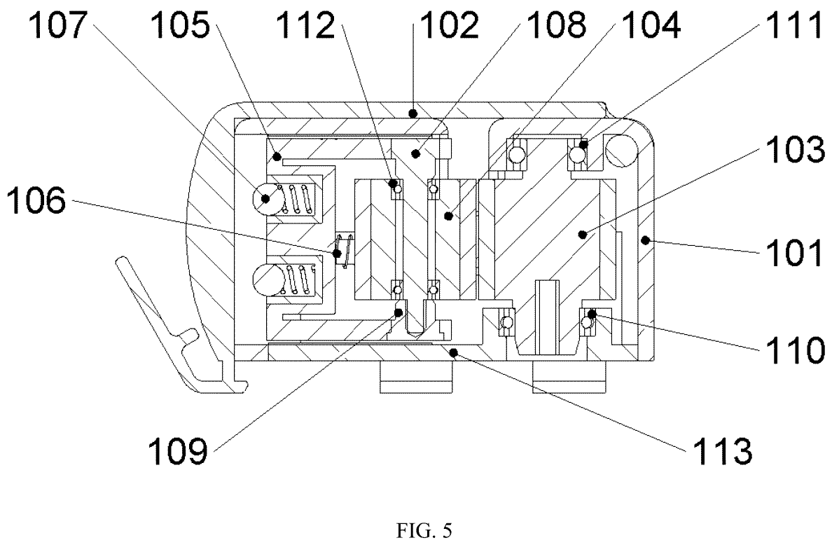

As shown in FIGS. 4 and 5, the guidewire catheter delivery module 1 further includes: the main body 101, the flip cover 102, the driven wheel frame 105, and the bottom cover 113. The bottom cover 113 is installed at the bottom of the main body 101, and the flip cover 102 is rotatably connected to the main body 101 through a shaft pin. The main body 101, the bottom cover 113, and the flip cover 102 are surrounded to form a cavity. The driven wheel frame 105, the driving wheel 103 and the driven wheel 104 are installed in the cavity. The driving wheel 103 is installed on a side of the driven wheel frame 105, and the driven wheel 104 is installed inside the driven wheel frame 105. The driven wheel frame 105 is allowed to drive the driven wheel 104 to move relative to the driving wheel 103 and the main body 101. When the flip cover 102 rotates away from the main body 101 through the shaft pin to be opened, the guidewire catheter 3 is allowed to be installed in the main body 101. When the flip cover 102 rotates towards the main body 101 through the shaft pin to be closed, the flip cover 102 limits the movement of the guidewire catheter 3. The driven wheel 104 is rotatably connected to the driven wheel frame 105, and the driven wheel 104 is allowed to move towards or away from the driving wheel 103 relative to the driven wheel frame 105. The driven wheel pressing ball 107 is installed on the side, facing away from the driving wheel 103, of the driven wheel frame 105, and the driven wheel pressing spring 115 is installed between the driven wheel 104 and the driven wheel pressing ball 107. When the flip cover 102 is closed, the driven wheel frame 105 is pressed by the flip cover 102 to move towards the driving wheel 103, and the driven wheel pressing spring 115 presses the driven wheel 104 towards the driving wheel 103. The limit block 114 is installed on a side wall of the driven wheel frame 105. When the driven wheel frame 105 moves away from the driving wheel 103, the limit block 114 interferes with the main body 101, and the driven wheel frame 105 limits the distance away from the driving wheel 103 through the limit block 114. A boss is provided on the side, adjacent to the driving wheel 103, of the inner wall of the main body 101, and the driven wheel release spring 106 is installed between the limit block 114 and the boss. When the flip cover 102 is opened, the driven wheel frame 105 moves away from the driving wheel 103 through the driven wheel release spring 106.

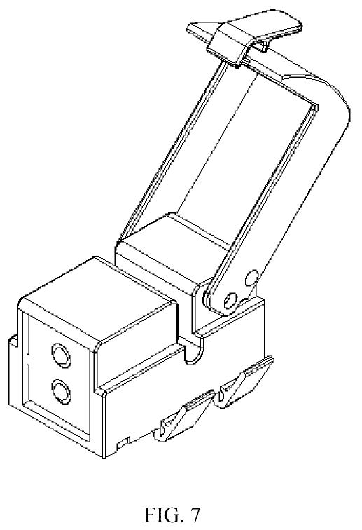

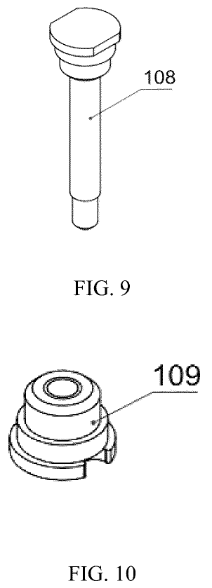

As shown in FIGS. 7, 9 and 10, buckles are provided on the sides, facing the motor drive and force feedback module 2, of the flip cover 102 and the bottom cover 113, and the buckles are clamped on the motor support frame 201. The driven wheel 104 is installed on the driven wheel frame 105 by the screw 108 and the nut 109, the driven wheel 104 is allowed to rotate around the screw 108, and the screw 108 and nut 109 are allowed to move relative to the main body 101. The first bearing 110 and the second bearing 111 are installed between the driving wheel 103 and the main body 101, and the third bearing 112 is installed between the driven wheel 104 and the screw 108.

### Embodiment 2

As shown in FIGS. 1 to 3, this embodiment includes: the guidewire catheter delivery module 1 and the motor drive and force feedback module 2. The guidewire catheter delivery module 1 is installed on the motor drive and force feedback module 2 and driven by the motor drive and force feedback module 2. The driving wheel 103 and the driven wheel 104 are installed in the guidewire catheter delivery module 1. The force sensor 202 and the motor 203 are installed in the motor drive and force feedback module 2, and an output end of the motor 203 is drivingly connected to the driving wheel 103. When the guidewire catheter 3 is pushed, the driving wheel 103 and the driven wheel 104 rotate to push the guidewire catheter 3 to move, and the guidewire catheter 3 generates a reverse force on the driving wheel 103 and the motor 203. The motor support frame 201 forms a lever through the support shaft 204, and the force sensor 202 generates a signal of a tensile force opposite to the force exerted on the motor 203.

As shown in FIGS. 4 and 5, the guidewire catheter delivery module 1 includes: the main body 101, the flip cover 102, the driving wheel 103, the driven wheel 104, the driven wheel frame 105, the driven wheel release spring 106, the driven wheel pressing ball 107, the screw 108, the nut 109, the first bearing 110, the second bearing 111, the third bearing 112, and the bottom cover 13. After the main body 101 and the flip cover 102 are connected through a shaft pin, the flip cover 102 can be opened to facilitate the placement or removement of the guidewire catheter 3. The driving wheel 103 is fixed on the main body 101 through the first bearing 110 and the second bearing 111, and the driven wheel 104 is fixed on the driven wheel frame 105 through the third bearing 112, the screw 108, and the nut 109.

As shown in FIGS. 6 to 10, the driven wheel release spring 106 and the driven wheel pressing ball 107 are fixed on the driven wheel frame 105. The driven wheel pressing ball 107 is installed on the side, facing away from the driving wheel 103, of the driven wheel frame 105. The driven wheel pressing spring 115 is installed between the driven wheel 104 and the driven wheel pressing ball 107. A boss is provided on the side, adjacent to the driving wheel 103, of the inner wall of the main body 101, the driven wheel release spring 106 is installed between the limit block 114 and the boss, and the limit block 114 is installed on the side wall of the driven wheel frame 105. When the driven wheel frame 105 moves away from the driving wheel 103, the limit block 114 interferes with the main body 101, the driven wheel frame 105 limits the distance away from the driving wheel 103 through the limit block 114, and the driven wheel release spring 106 and the driven wheel pressing spring 115 can control the driven wheel 104 to tightly press or release the guidewire catheter 3. The bottom cover 113 is fixedly connected to the main body 101.

The force sensor 202 and the motor 203 are fixed on the motor support frame 201. Two support shafts 204 are provided on the side of the motor support frame 201 and are rotatably installed on the fixed seat 205. The guidewire catheter delivery module 1 is fixed on the motor drive and force feedback module 2 through a buckle, and the entire guidewire catheter delivery module 1 can be released by pressing the buckle on one side. The flip cover 102 can also be fixed on the motor drive and force feedback module 2 through a buckle, and can also be released by a button. The D-shaped shaft of the motor 203 is matched with the D-shaped hole in the driving wheel 103, and after connection, the motor 203 can drive the driving wheel 103 to rotate. The flip cover 102 can be opened and locked at any time, and can be used for positioning, loading and unloading the guidewire catheter 3 at the same time. When the flip cover 102 is closed, the upward and downward movement of the guidewire catheter 3 is limited. When the flip cover 102 is opened, the guidewire catheter 3 can be put in or taken out from above. When the flip cover 102 is opened, the internal driven wheel release spring 106 pushes out the driven wheel 104, and the guidewire catheter 3 is released. When the flip cover 102 is closed, the external driven wheel pressing spring 115 presses the driven wheel 104 tightly, so that the guidewire catheter 3 is clamped tightly, and finally the motor 203 can drive the advancement or retraction of the guidewire catheter 3. The driven wheel frame 105 and the driven wheel 104 are assembled by the screw 108 and the nut 109 to translate within the main body 101. When the guidewire catheter 3 moves forward and backward, the motor support frame 201 tends to swing, which sequentially generates a pulling force or pressing force on the force sensor 202 installed at the bottom. The force sensor 202 at the bottom includes one end connected to the motor support frame 201, and the other end connected to the fixed seat 205 to ensure that the pulling force or pressing force can be generated.

Those skilled in the art should know that, in addition to realizing the system and its various devices, modules and units provided by the present invention in a purely computer-readable program code form, through logical programming of the method steps, the system and its various devices, modules and units provided by the present invention can realize the same functions in the form of logic gates, switches, application-specific integrated circuits, programmable logic controllers, embedded microcontrollers, etc. Therefore, the system and its various devices, modules and units provided by the present invention can be regarded as a hardware component, and the devices, modules and units included therein for realizing various functions can also be regarded as structures within the hardware component; or the devices, modules and units for realizing various functions can also be regarded as either software modules for implementing methods or structures within hardware components.

In the description of the present application, it should be understood that the orientations or positional relationships indicated by the terms “upper”, “lower”, “front”, “rear”, “left”, “right”, “vertical”, “horizontal”, “top”, “bottom”, “inner”, “outer”, etc. are based on the orientations or positional relationships shown in the drawings, are only for convenience in describing the present application and simplifying the description, and do not indicate or imply that the devices or elements referred to must have a specific orientation or be constructed and operated in a specific orientation. Therefore, they cannot be construed as a limitation on the present application.

Compared with the prior art, the present invention has the following beneficial effects:
1. The present invention is practical since it can be used in a surgical robot for intervention to avoid surgical radiation and improve surgical safety;
2. The present invention realizes the delivery of the guidewire catheter and has high stability;
3. The device provided by the present invention has a compact structure and saves space.

Specific embodiments of the present invention have been described above. It should be understood that the present invention is not limited to the specific embodiments described above, and those skilled in the art can make various changes or modifications within the scope of the claims without affecting the essence of the present invention. On the premise of no conflict, the embodiments of the present application and features in the embodiments may be arbitrarily combined with each other.

What is claimed is:

1. A guidewire catheter delivery device, comprising a guidewire catheter delivery module and a motor drive and force feedback module;
the motor drive and force feedback module is drivingly connected to the guidewire catheter delivery module, and the guidewire catheter delivery module is installed with a guidewire catheter and drives the guidewire catheter to move back and forth;
a force sensor and a motor are installed in the motor drive and force feedback module;
an output end of the motor is connected to the guidewire catheter delivery module, and an end, facing away from the guidewire catheter delivery module, of the motor is connected to the force sensor;
a driving wheel and a driven wheel are installed in the guidewire catheter delivery module; and
the driving wheel is connected to the output end of the motor, and the guidewire catheter is installed between the driving wheel and the driven wheel.

2. The guidewire catheter delivery device according to claim 1, wherein the motor drive and force feedback module further comprises: a motor support frame and a support shaft;
the force sensor and the motor are installed in the motor support frame, the output end of the motor extends out of a first end of the motor support frame, and the force sensor is installed at a second end of the motor support frame; and
the support shaft extends outward from a middle of the motor support frame.

3. The guidewire catheter delivery device according to claim 2, wherein the motor support frame is installed in a fixed seat;
the motor support frame is rotatably connected to the fixed seat through the support shaft;
the fixed seat is fixedly connected to the force sensor; and
when the guidewire catheter moves, the motor receives a reaction force from the guidewire catheter, the first end, adjacent to the guidewire catheter delivery module, of the motor support frame receives the reaction force, and the second end, connected to the force sensor, of the motor support frame receives a force that is opposite to the reaction force through the support shaft.

4. The guidewire catheter delivery device according to claim 2, wherein the guidewire catheter delivery module further comprises a main body, a flip cover, a driven wheel frame, and a bottom cover;
the bottom cover is installed at a bottom of the main body, and the flip cover is rotatably connected to the main body through a shaft pin; the main body, the bottom cover, and the flip cover are surrounded to form a cavity;
the driven wheel frame, the driving wheel, and the driven wheel are installed in the cavity;
the driving wheel is installed on a side of the driven wheel frame, and the driven wheel is installed inside the driven wheel frame;
the driven wheel frame is allowed to drive the driven wheel to move relative to the driving wheel and the main body;
when the flip cover rotates away from the main body through the shaft pin to be opened, the guidewire catheter is allowed to be installed in the main body; and
when the flip cover rotates towards the main body through the shaft pin to be closed, the flip cover limits movement of the guidewire catheter.

5. The guidewire catheter delivery device according to claim 4, wherein the driven wheel is rotatably connected to the driven wheel frame, and the driven wheel is allowed to move towards or away from the driving wheel relative to the driven wheel frame;
a driven wheel pressing ball is installed on a side, facing away from the driving wheel, of the driven wheel frame, and a driven wheel pressing spring is installed between the driven wheel and the driven wheel pressing ball; and
when the flip cover is closed, the driven wheel frame is pressed by the flip cover to move towards the driving wheel, and the driven wheel pressing spring presses the driven wheel towards the driving wheel.

6. The guidewire catheter delivery device according to claim 4, wherein a limit block is installed on a side wall of the driven wheel frame; and
when the driven wheel frame moves away from the driving wheel, the limit block interferes with the main body, and the driven wheel frame limits a distance away from the driving wheel through the limit block.

7. The guidewire catheter delivery device according to claim 6, wherein a boss is provided on a side, adjacent to the driving wheel, of an inner wall of the main body, and a driven wheel release spring is installed between the limit block and the boss; and
when the flip cover is opened, the driven wheel frame moves away from the driving wheel through the driven wheel release spring.

8. The guidewire catheter delivery device according to claim 4, wherein buckles are provided on sides, facing the motor drive and force feedback module, of the flip cover and the bottom cover; and
the buckles are clamped on the motor support frame.

9. The guidewire catheter delivery device according to claim 4, wherein the driven wheel is installed on the driven wheel frame by a screw and a nut;
the driven wheel is allowed to rotate around the screw;
the screw and the nut are allowed to move relative to the main body; and
a first bearing and a second bearing are installed between the driving wheel and the main body, and a third bearing is installed between the driven wheel and the screw.

10. A robot, comprising the guidewire catheter delivery device according to claim 1.

11. The robot according to claim 10, wherein in the guidewire catheter delivery device, the motor drive and force feedback module further comprises: a motor support frame and a support shaft;
the force sensor and the motor are installed in the motor support frame, the output end of the motor extends out of a first end of the motor support frame, and the force sensor is installed at a second end of the motor support frame; and
the support shaft extends outward from a middle of the motor support frame.

12. The robot according to claim 11, wherein in the guidewire catheter delivery device, the motor support frame is installed in a fixed seat;
the motor support frame is rotatably connected to the fixed seat through the support shaft;
the fixed seat is fixedly connected to the force sensor; and
when the guidewire catheter moves, the motor receives a reaction force from the guidewire catheter, the first end, adjacent to the guidewire catheter delivery module, of the motor support frame receives the reaction force, and the second end, connected to the force sensor, of the motor support frame receives a force that is opposite to the reaction force through the support shaft.

13. The robot according to claim 11, wherein in the guidewire catheter delivery device, the guidewire catheter delivery module further comprises a main body, a flip cover, a driven wheel frame, and a bottom cover;
the bottom cover is installed at a bottom of the main body, and the flip cover is rotatably connected to the main body through a shaft pin; the main body, the bottom cover, and the flip cover are surrounded to form a cavity;
the driven wheel frame, the driving wheel, and the driven wheel are installed in the cavity;
the driving wheel is installed on a side of the driven wheel frame, and the driven wheel is installed inside the driven wheel frame;
the driven wheel frame is allowed to drive the driven wheel to move relative to the driving wheel and the main body;
when the flip cover rotates away from the main body through the shaft pin to be opened, the guidewire catheter is allowed to be installed in the main body; and
when the flip cover rotates towards the main body through the shaft pin to be closed, the flip cover limits movement of the guidewire catheter.

14. The robot according to claim 13, wherein in the guidewire catheter delivery device, the driven wheel is rotatably connected to the driven wheel frame, and the driven wheel is allowed to move towards or away from the driving wheel relative to the driven wheel frame;
a driven wheel pressing ball is installed on a side, facing away from the driving wheel, of the driven wheel frame, and a driven wheel pressing spring is installed between the driven wheel and the driven wheel pressing ball; and
when the flip cover is closed, the driven wheel frame is pressed by the flip cover to move towards the driving wheel, and the driven wheel pressing spring presses the driven wheel towards the driving wheel.

15. The robot according to claim 13, wherein in the guidewire catheter delivery device, a limit block is installed on a side wall of the driven wheel frame; and
when the driven wheel frame moves away from the driving wheel, the limit block interferes with the main body, and the driven wheel frame limits a distance away from the driving wheel through the limit block.

16. The robot according to claim 15, wherein in the guidewire catheter delivery device, a boss is provided on a side, adjacent to the driving wheel, of an inner wall of the main body, and a driven wheel release spring is installed between the limit block and the boss; and
when the flip cover is opened, the driven wheel frame moves away from the driving wheel through the driven wheel release spring.

17. The robot according to claim 13, wherein in the guidewire catheter delivery device, buckles are provided on sides, facing the motor drive and force feedback module, of the flip cover and the bottom cover; and
the buckles are clamped on the motor support frame.

18. The robot according to claim 13, wherein in the guidewire catheter delivery device, the driven wheel is installed on the driven wheel frame by a screw and a nut;
the driven wheel is allowed to rotate around the screw;
the screw and the nut are allowed to move relative to the main body; and
a first bearing and a second bearing are installed between the driving wheel and the main body, and a third bearing is installed between the driven wheel and the screw.
